(19) United States
(12) Patent Application Publication
(10) Pub. No.: US 2024/0390577 A1
(43) Pub. Date: Nov. 28, 2024

(54) PUMP MECHANISM FOR MEDICATION DELIVERY AND MEDICATION DELIVERY SYSTEM

(71) Applicant: Becton, Dickinson and Company, Franklin Lakes, NJ (US)

(72) Inventors: Alessandro PIZZOCHERO, Chelmsford, MA (US); Mark WOOD, Sterling, MA (US)

(73) Assignee: Becton, Dickinson and Company, Franklin Lakes, NJ (US)

(21) Appl. No.: 18/694,137

(22) PCT Filed: Sep. 23, 2022
(86) PCT No.: PCT/US2022/044554

§ 371 Date: Mar. 21, 2024

Related U.S. Application Data
(60) Provisional application No. 63/248,145, filed on Sep. 24, 2021.

(51) Int. Cl. A61M 5/142; A61M 5/145; A61M 5/315

(52) U.S. Cl. CPC A61M 5/14248; A61M 5/1452; A61M 2005/14268; A61M 2005/31518

(57) ABSTRACT

Drug delivery device, drug delivery system, and system components are provided including configuration of a container, reservoir or barrel for medium or fluid and a pump mechanism or driving components for advancing a plunger to dispense the medium or fluid from the reservoir or barrel, and/or for filling the reservoir or barrel with the medium or fluid, where the pumping mechanism or driving components are disposed such that individual and/or overall dimensions of the pump mechanism or the driving components can be reduced compared to conventional designs. A provided system can include a syringe-style drug container, reservoir, or barrel containing a medium or fluid which can be dispensed by a pumping mechanism configured to advance a plunger/stopper disposed inside the barrel via cable, or a ribbon, connected to the plunger/stopper.

### BACKGROUND

Generally, exemplary embodiments of the present disclosure relate to the fields of medication delivery devices. More specifically, exemplary embodiments of the present disclosure relate to medication delivery devices where a stopper or plunger is advanced through a reservoir to dispense medication from the reservoir.

Medication delivery devices of the present disclosure can be useful in the field of insulin therapy, for example for the treatment of type 1 diabetes. One method of insulin therapy includes syringes and insulin pens that require a needle stick at each injection, typically three to four times per day that are simple to use and relatively low in cost. Another widely adopted and effective method of treatment for managing diabetes is the use of an insulin pump. Insulin pumps can help the user keep blood glucose levels within target ranges based on individual needs, by continuous infusion of insulin.

In the example of medical applications where medication delivery devices of the present disclosure can be particularly useful is patch pumps. A patch pump is an integrated device that facilitates infusion therapy for diabetic patients. A patch pump combines most or all of the fluidic components, including the fluid reservoir, pumping mechanism and mechanism for automatically inserting the cannula, in a single housing which is adhesively attached to an infusion site on the patient's skin, and does not require the use of a separate infusion or tubing set. A patch pump containing insulin adheres to the skin and delivers the insulin over a period of time via an integrated subcutaneous cannula. Some patch pumps may be configured to include wireless communication with a separate controller device, while others are completely self-contained. Such devices are replaced on a frequent basis, such as every three days, particularly when the insulin reservoir is exhausted.

As patch pumps are designed to be a self-contained unit that is worn by the diabetic patient, it is preferable to be as small as possible so that it does not interfere with the activities of the user. Thus, in order to minimize discomfort to the user, it would be preferable to minimize the overall size of the patch pump. Conventional patch pumps or a syringe-type devices typically include a driving or pump mechanism with a single advancing lead screw inside medium or fluid reservoir or chamber to push, advance, or otherwise apply force on the plunger in order to dispense the medium or fluid out of the chamber.

In order to minimize the size of medication delivery devices, such as a patch pump, the design of the device and/or design of its constituent parts, such as those of the driving or mump mechanisms, should be reduced as much as possible without compromising the accuracy and reliability of the device or its feature set.

### SUMMARY

Exemplary embodiments of the disclosure may address at least the above problems and/or disadvantages and other disadvantages not described above. Also, exemplary embodiments are not required to overcome the disadvantages described above, and may not overcome any of the problems described above.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

As would be readily appreciated by skilled artisans in the relevant art, while descriptive terms such as “medium”, “medicament”, “stopper”, “plunger”, “thread”, “syringe”, “motor”, “bridge”, “nut”, “gear”, “wall”, “top”, “side”, “bottom,” “upper,” “lower,” “proximal”, “distal”, “container”, “reservoir”, “chamber”, “cable”, and others are used throughout this specification to facilitate understanding, it is not intended to limit any components that can be used in combinations or individually to implement various aspects of the embodiments of the present disclosure.

Exemplary embodiments of the present disclosure provide system components that can facilitate a reduction in the overall size or footprint of a drug delivery device, such as a patch pump, by a configuration of a container, reservoir or barrel for medium or fluid and a pump mechanism or driving components for advancing a plunger to dispense the medium or fluid from the reservoir or barrel, and/or for filling the reservoir or barrel with the medium or fluid, where the pumping mechanism or driving components can be disposed such that individual and/or overall dimensions of the pump mechanism or the driving components can be reduced compared to conventional designs.

Exemplary implementations of exemplary embodiments of the present disclosure provide various feature and component which may be deployed individually or in various combinations.

According to exemplary embodiments of the present disclosure, a system includes a syringe-style drug container, reservoir, or barrel containing a medium or fluid which can be dispensed by a pumping mechanism configured to advance a plunger/stopper disposed inside the barrel via cable, or a ribbon, connected to the plunger/stopper.

According to another exemplary embodiment of the present disclosure, a driving mechanism based on a syringe barrel body is provided, for example for use in a medication delivery system, such as a patch pump, where a plunger can be advanced axially with respect to a barrel by a cable, or a ribbon, connected to the plunger/stopper and driven by, for example, a motor through a gearing as appropriate for a desired application to dispense the medium or fluid out of the barrel.

According to exemplary embodiments of the present disclosure, significant space savings can be achieved by utilizing exemplary implementations of a pumping mechanism, including for example cable-driven pump configurations as provided in the exemplary embodiments of the present disclosure.

### BRIEF DESCRIPTION OF THE DRAWINGS

The above and/or other example aspects and advantages will become apparent and more readily appreciated from the following description of example embodiments, taken in conjunction with the accompanying drawings in which:

FIGS. 1A and 1B are examples of perspective views of an exterior of a device according to exemplary embodiments of the present disclosure.

FIGS. 2A and 2B diagrammatically illustrate a combination of system components according to exemplary embodiments of the present disclosure.

FIGS. 2C, 2D, and 2E diagrammatically illustrate examples of configurations of certain components according to embodiments of the present disclosure.

FIG. 3A shows a perspective three dimensional view of a configuration of certain components in a device according to exemplary embodiments of the present disclosure.

FIG. 3B shows an enlarged view and details of a portion of FIG. 3A.

FIGS. 4A, 4B, and 4C show respectively a front view, a top view, and a perspective three dimensional back view of configuration of certain components in a device according to exemplary embodiments of the present disclosure.

FIG. 5A shows a perspective three dimensional view of a configuration of certain components in a device according to exemplary embodiments of the present disclosure.

FIGS. 5B and 5C illustrate details of certain components of a device according to exemplary embodiments of the disclosure.

FIG. 6A illustrates an example of a perspective view of a component of a device according to an exemplary implementation of exemplary embodiments of the disclosure.

FIG. 6B shows an enlarged view and details of a portion of FIG. 6A.

FIGS. 7A and 7B diagrammatically illustrate a combination of system components according to further exemplary embodiments of the present disclosure.

FIG. 8A shows a perspective three dimensional view of a configuration of certain components in a device according to further exemplary embodiments of the present disclosure.

FIG. 8B shows a perspective three dimensional bottom view of a configuration of certain components of FIG. 8A.

FIGS. 9A and 9B diagrammatically show certain components of devices according to exemplary implementations of exemplary embodiment of the disclosure, and examples of mathematical computations related to features thereof.

FIG. 10A shows a perspective three dimensional view of a configuration of certain components of devices according to exemplary embodiments of the present disclosure.

FIG. 10B shows another perspective three dimensional view and details of FIG. 10A.

FIG. 10C illustrates in a two dimensional view a configurations of certain features of one of the components of FIGS. 10A and 10B.

FIG. 11A shows a top view of a configuration of certain components in a device according to further exemplary embodiments of the present disclosure.

FIG. 11B shows an enlarged perspective three dimensional view and details of a portion of a device illustrated in FIG. 11A.

FIGS. 12A and 12B show respectively perspective three dimensional back and front views of a device illustrated in FIG. 11A.

### DETAILED DESCRIPTION OF EXEMPLARY EMBODIMENTS

Referring now to the drawings, wherein like reference numerals designate identical or corresponding parts throughout the several views, embodiments of the present disclosure are described as follows.

It will be understood that the terms “include,” “including,” “comprise,” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be further understood that, although the terms “first,” “second,” “third,” etc., may be used herein to describe various elements, components, regions, layers and/or sections, these elements, components, regions, layers and/or sections may not be limited by these terms. These terms are only used to distinguish one element, component, region, layer or section from another element, component, region, layer or section.

As used herein, the term “and/or” includes any and all combinations of one or more of the associated listed items. Expressions such as “at least one of,” when preceding a list of elements, modify the entire list of elements and do not modify the individual elements of the list. In addition, the terms such as “unit,” “-er (-or),” and “module” described in the specification refer to an element for performing at least one function or operation, and may be implemented in hardware, software, or the combination of hardware and software.

Various terms are used to refer to particular system components. Different companies may refer to a component by different names—this document does not intend to distinguish between components that differ in name but not function.

Matters of these exemplary embodiments that are obvious to those of ordinary skill in the technical field to which these exemplary embodiments pertain may not be described here in detail. In addition, various features of the exemplary embodiments can be implemented individually or in any combination or combinations, and would be understood by one of ordinary skill in the art of medicament delivery devices.

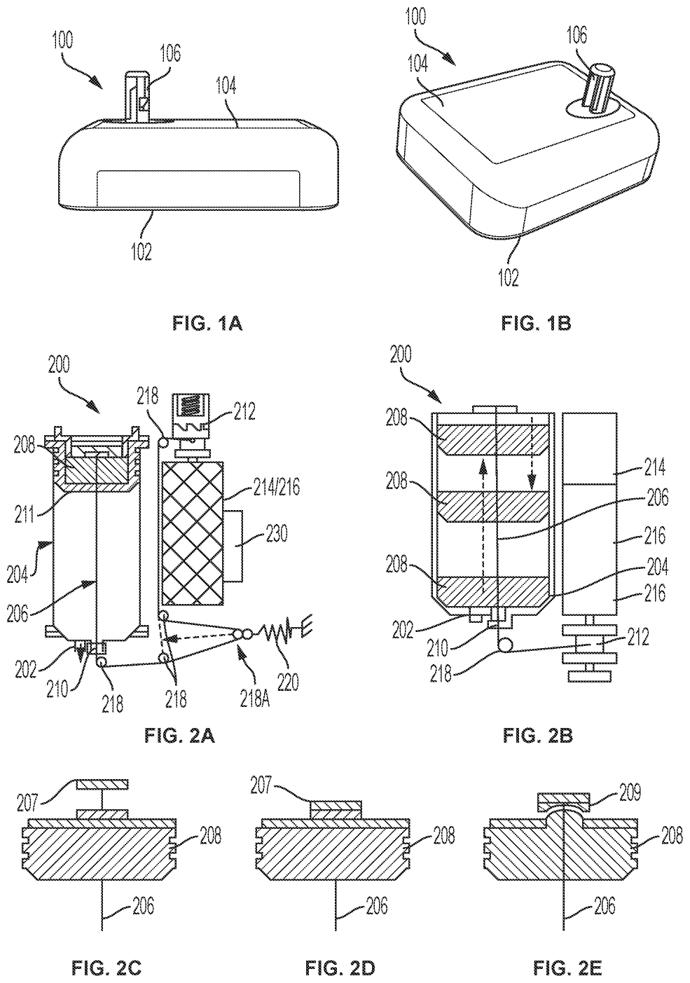

Exemplary embodiments of the present disclosure can be applied to a pump concept, such as for example a wearable disposable patch pump 100 configured to include a base 102, outer housing 104, and an insertion mechanism 106, as shown in perspective views of FIGS. 1A and 1B.

Referring to FIGS. 2A-2E, 3A, 3B, 4A-4C, and 5A-5C, exemplary embodiments of the disclosure provide a cable-driven pump 200 where the pump is comprised of an output 202 (which can be connected to a syringe or an insertion mechanism) and barrel or reservoir 204 of any appropriate cross-section shape and in fluid communication with output 202, and a cable (or ribbon) 206 is connected either fixedly to plunger/stopper 208, such as insert molded (and fully enclosed by the elastomeric stopper, as illustrated in the example of FIG. 2A), or slidably with respect to the plunger/stopper 208 (as illustrated in the examples of FIGS. 2C and 2E). Cable 206 extends from proximal surface of 211 of plunger/stopper 208 can exit barrel/reservoir 204 via a septum 210 at a proximal end (dispense side) of barrel/reservoir 204, and can be connected to a spool 212 driven by a motor 214/gearbox 216, for example to roll cable 206 onto spool 212, thereby advancing plunger/stopper 208 with respect to barrel/reservoir 204. Operational control of pump 200 can be provided by electronics 230 and/or 220, which can be disposed, for example, on a base 102.

According to exemplary implementations, spool 212 can be located near a distal end of barrel/reservoir 204 (as shown in the non-limiting illustration of FIG. 2A), or can be located near a proximal end of barrel/reservoir 204 (as shown in the non-limiting diagrammatical illustration of FIG. 2B). In yet further exemplary implementation, one or more pivot pin(s) or pulley(s) 218, or any combinations thereof, can be provided between septum 210 and spool 212 to guide, and/or control tension of, cable 206. In still further exemplary implementation one or more pulley(s) 218A, can be, for example, moved or positioned to adjust tension of cable 206, where the tension can be measured 220.

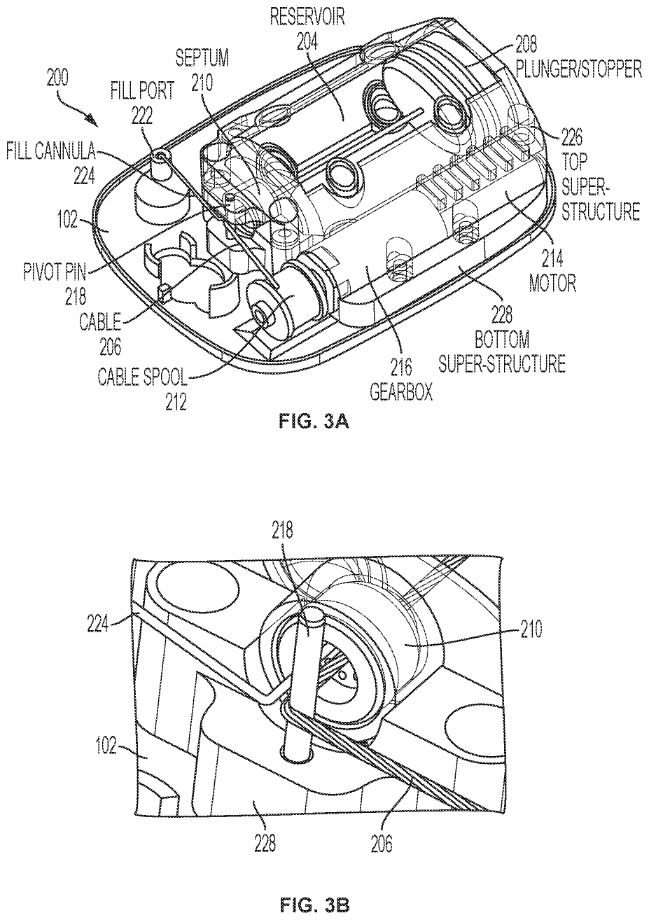

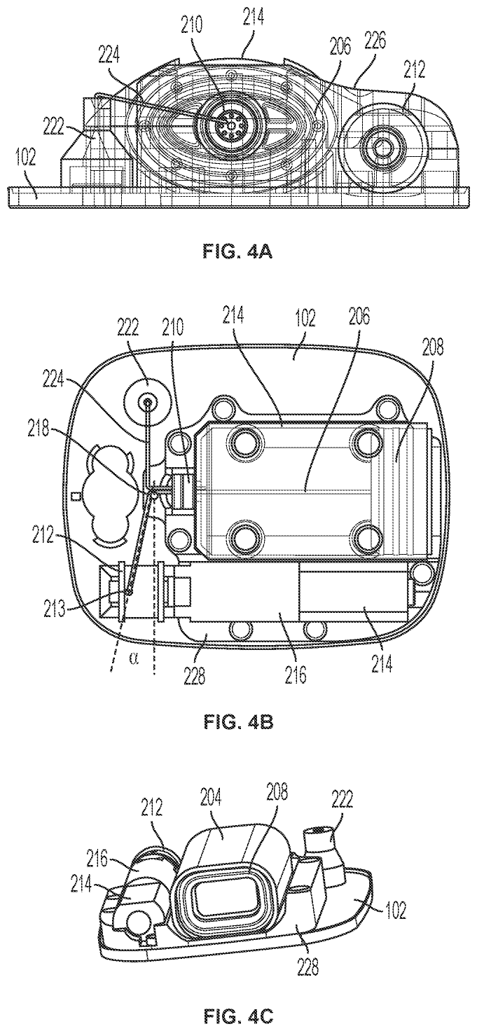

FIG. 3A illustrates in a perspective view an example of a configuration of at least some of the components according to an exemplary implementation of a medication delivery system 200, such as on a base 102 of a wearable disposable patch pump 100, of disclosed exemplary embodiments. As shown in the example of FIG. 3A, reservoir 204 can be connected to a fill port 222 via fill cannula 224 extending into reservoir 204 via septum 210. In addition, a top super-structure 226 and a bottom superstructure 228 can be provided on a base, such as base 102, to support, connect, and/or cover various components disposed thereon. FIGS. 4A and 4B provide diagrammatic front and top view, respectively, of an exemplary implementation of exemplary embodiment illustrated in FIG. 3A. In an exemplary implementation, system or pump 200 can be for one-time, disposable use, and may be filled at time of use, or come pre-filled.

According to an exemplary implementation, as illustrated in a more detailed view of FIG. 3B, cable 206 runs, through a septum 210 at the end of the barrel/reservoir 204 (the dispense side) and is then routed around a fixed or rotating pin/spindle 218 to a spool/drum or rotary/linearly-driven component 212 (attached to a motor 214/gearbox 216) that will pull said cable 206 for a distance equal to the desired plunger/stopper 208 travel. The septum 210 is configured such that cable 206 can move outside of the inner, drug-filled volume of the barrel/reservoir 204, while not allowing any fluid to leak. As illustrated in FIG. 3B, cable 206 can be a braded cable bent around pivot pin 218.

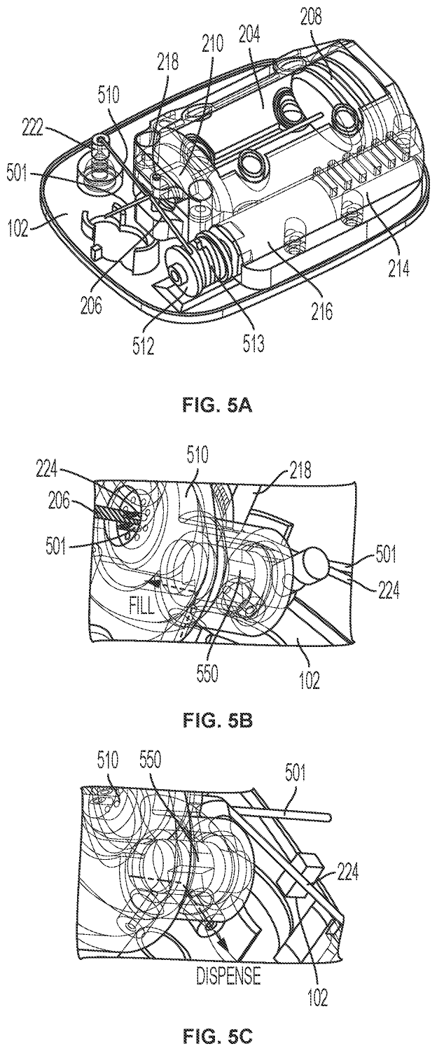

FIG. 5A illustrates in a perspective view a further example of a configuration of at least some of the components according to an exemplary implementation of a medication delivery system, such as system or pump 200, including a delivery or output configuration comprising a needle or cannula 501 inserted into septum 510 to be in liquid communication with interior of barrel/reservoir 204, as illustrated in more detailed view of FIGS. 5B and 5C.

According to an exemplary implementation as illustrated in FIGS. 5B and 5C, a fill/dose plug/valve 550 can be provided to facilitate switching between filling operation and dose dispensing operation of pump 200. An example of a filling position of valve 550 is shown in FIG. 5B. In an exemplary implementation, valve 550 can be of a molded hard plastic with an elastomer, for example fully encompassing the hard structure, to facilitate sealing and having the same V-notch for flow. An example of a dispensing position of valve 550 is shown in FIG. 5C.

According to an exemplary implementation, cable 206 interfaces with the plunger/stopper 208 (either insert-molded, or attached during assembly), in a manner that the stopper sealing ability is not compromised, as illustrated in the examples of FIGS. 2C and 2D. Cable 206 may be made from individual or multiple braided strands (as illustrated in the example of FIG. 3A) and may have varying mechanical properties along its length such as design features found in guidewires. For example, materials can be high stiffness/strength with low flexural modulus (braiding supports this combination), and may be metal or plastic. Cable 206 may be permanently coated with a suitably bio-compatible material that is also appropriate for sliding and sealing through septum 210 and/or plunger/stopper 208, withstand sterilization and aging, and can fold around guide pins/spindles 218. In exemplary implementations, cable 206 can be sufficiently strong to mechanically withstand termination at either end and safely withstand tight bends at mounting points (such as at connection to spool 212). High precision/resolution in delivery volumes can be achieved with stiff cables that can be bent over spools of suitable size to fit into a small device. According to exemplary implementations, cable 206 may also be in the form of a ribbon. A ribbon configuration can have a greater margin from plastic deformation and/or possibly facilitate improved management thereof at spooling. As in the case of cable configuration, leakage at septum 210 and stopper/plunger 208 should be avoided.

In exemplary implementations applicable to any combinations of disclosed exemplary embodiments, cable 206 design can be tailored to avoiding any potential leakage through any of the elastomeric interfaces (plunger/stopper 208 on distal end, and septum 210 at proximal end). For example, special cable designs and coatings can be used, and alternatively or in addition, properly tuning the elastomer materials through durometer, and geometry of supporting and/or assembly parts that favor increased compression around the cable.

In an exemplary implementation, a motor 214/gearbox 216 turns to pull cable 206 to a desired amount to pull the plunger/stopper 208 and thus deliver fluid out of barrel/reservoir 204. It should be noted that the movement of a point on cable 206 at the motor 214/gearbox 216 end may not be the same as a point in the syringe as there are different intermediate forces and thus a different amount of strain that can be experienced by cable 206 at different locations. According to exemplary implementations, such behavior can be compensated for by operating in non-plastic regime for the cable extension. According to other exemplary implementations, operation in plastic regime can be achieved, but may require more sophisticated drive algorithms and/or may provide a lower resolution.

According to exemplary implementations, motor 214/gearbox 216 subsystem may be modified to include a cable spindle/drum, including for example spool 212. In an exemplary implementation where cable 206 can be permanently affixed to plunger/stopper 208, a design of spindle/drum for the cable pull can be presented in a manner that filling may be accomplished freely, and upon driving motor 214 a mechanism engages spool 212 to pull cable 206. As noted, in exemplary implementations, such a mechanism can be built into the motor 214/gearbox 216 sub-assembly.

According to exemplary implementations, balancing of motor/gearbox output torque and required drive torque can be affected by material choices, geometry of components, electrical drive circuit, motor/gearbox properties, and general layout. FIGS. 2A through 5C diagrammatically illustrate representative non-limiting geometry of exemplary implementations of various components according to embodiments of the disclosure. For example, an exemplary implementation of a large spindle of spool 212 for spooling of cable 206 may impose a greater torque on motor 214 on the one hand, but may reduce risk of overlapping spooled cable 206 on the other hand. Conversely, a smaller spool 212 may correspond to lower torque, however if not properly size, cable 206 may overlap itself once spooled on spool 212, thus potentially influencing dose accuracy. Exemplary implementations of disclosed embodiments provide features that can facilitate control of such factors, including for example, providing spool 512 with grooves 513 to facilitate control of cable 206 position as it is turned and pulled by motor 214. As diagrammatically illustrated in the example of FIG. 4B, cable 206 can be attached to spool 212 at an initial angle α to facilitate rolling of cable 206 onto spool 212. According to an exemplary implementation, dose accuracy can depend on angle α, such that the large the angle α, the larger the error on dose accuracy, and as cable 206 is spooled or rolled onto spool 212, the error reduces if cable 206 goes closer to the straight line (angle α=0) to the pivot 218. According to exemplary implementations, minimizing angle α that cable 206 makes from the pivot pin 218 to the spooling contact point can reduce dose error.

In an exemplary non-limiting implementation, additional sources of torque may be due to stopper/plunger 208 friction forces against the inner surface of the barrel/reservoir 204. Stopper 208 percent compression as well as contact area and pressures, together with materials can factor into overall friction force. For example, the larger the area the greater the friction. This interface can however be also responsible for guaranteeing leak resistance during motion and a proper balance should be achieved to facilitate proper operation. According to exemplary implementations, a potential further consideration can be a difference between static and dynamic friction (for example, depending on pumping rate and frequency) as well as initial friction.

According to exemplary implementations, barrel/reservoir 204 may be of circular, elliptical, or generally rectangular shape. In a non-limiting exemplary implementation, as diagrammatically illustrated in FIG. 4C showing an example of a perspective back (distal end) view of some of the components of pump 200, barrel/reservoir 204 can be provided with a more rectangular cross-section for example to minimize space and maximize transverse volume available for other components (for example, a space savings of can be achieved on each side of barrel/reservoir 204). According to further non-limiting implementations, barrel/reservoir 204 can be any non-symmetric shaped designed to optimize space utilization in the device. Special design consideration should be made to ensure the seal is maintained as less “smooth” shapes are employed for space saving. Any non-symmetry in the cross-section shape may result in offset pulling forces on plunger/stopper 208 that may lead to non-planar motion thus adding to individual stroke dose error.

According to exemplary implementations, a potential further consideration is cable 206 stiffness being sufficient such that when it is pulled, it does not deform to an extent that dose volume is affected. For example, stretch of a cable 206 can occur and when the stress is below yield and the cable stays in the elastic operating range, then according to an exemplary implementation a compensation may be made to ensure consistent dosing accuracy—for example, mathematical equations of a system can show such relationship and may be adapted to a suitable control system 220 and/or 230, for example configured to provide specific, rather than generic, control tailored to specific system parameters. In an exemplary implementation, as cable 206 is pulled, the free length off the spool 212 can be reduced and the amount of stretch can also be reduced, thus improving control quality, as diagrammatically illustrated for example in FIG. 2A.

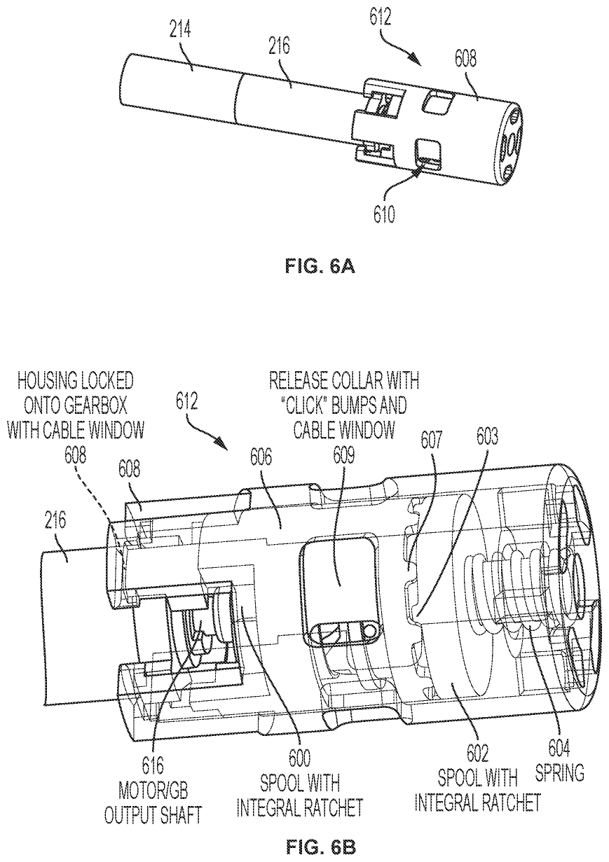

According to another exemplary implementation, cable can be continuous (for example, forming a closed loop), and driven by a linear ratcheting mechanism or by a suitably configured spool with an integral ratchet, as shown for example in FIGS. 6A and 6B, which can be implemented in any exemplary configurations of exemplary embodiments of a pump disclosed herein. An exemplary embodiment provides a ratchet mechanism for cable de-spooling and/or spooling that can be configured in exemplary implementations of pump mechanism and medication delivery system according to the disclosure. FIG. 6A illustrates an example of a motor 214/gearbox 216 with attached external de-spooling/spooling ratchet mechanism 612 for filling according to an exemplary implementation. As illustrated in a more detailed view in FIG. 6B, mechanism 612 can comprise a housing 608 locked onto a gearbox 216, a first ratchet component 600 in communication with (for example, rotationally locked with) shaft 616 of motor 214/gear 216, a second ratchet component 602 in communication with the first ratchet component 600 (for example first and second components are biased by a spring 604), a release collar 606 having click protrusions (bumps or teeth) 607 in communication with second ratchet component 602, for example via corresponding protrusions (bumps or teeth) 603 of component 602, and a cable window 609 providing access for cable 206 for connection to integral ratchet 610.

According to an exemplary implementation of a spooling/de-spooling mechanism 612, in a system such as a system or pump 200, can facilitate stability of cable 206 with respect to plunger/stopper 208. For example, configuration of gearbox 216 and ratchet components 600/602 can help ensure that cable 206 does not have to move with respect to the plunger 208.

According to further exemplary implementations, deploying a spooling/de-spooling mechanism 612 in a system such as system or pump 200 can facilitate sealing of the cable 206 to the plunge/stopper 208 in a manner that potential leak pathways can be fully occluded. This can be particularly significant for a braided cable system where leak management through the small braids may be more challenging. Alternatively, or in combination with any disclosed implementation, a configuration of the plunger/stopper and cable as illustrated in FIG. 2E and described herein can also significantly mitigate potential leak between a cable and a plunger/stopper.

According to exemplary implementations, a termination can be required in order to drive the plunger/stopper motion. Such an exemplary implementation can incorporate a design balancing the various sections of cable 206 that may be under varying forces between the various segment interfaces such as, for example, (1) plunger/stopper 208 to septum 210, (2) septum 210 to a pivot pin 218, (3) a pivot pin 218 to a pulley 218A, (4) a pulley 218A or a pivot pin 218 to a driver including spool 212/512/612, motor 214, gear 216, and/or (5) other segments. When cable 206 is exposed to different forces between various interfaces the total stretch may be different and thus non-uniform tension may occur. In yet further exemplary implementations, tensioners (for example, unit 220) may be employed to counter such effects.

Various configurations comprising any combinations of exemplary implementations and/or features disclosed herein can be implemented without departing from the teachings of the present disclosure. An exemplary non-limiting benefit that may be achieved from such exemplary arrangements of component and/or features is that a very linear pump may be provided.

Furthermore, various configurations comprising any combinations of exemplary implementations and/or features disclosed herein can be implemented to facilitate filling of a medication dispensing system according to exemplary embodiments of the disclosure.

According to an exemplary non-limiting implementation of a filling feature, cable 206 can run through, slide, and seal on plunger/driver 208. For example, a termination 207 of cable 206 can be held behind the syringe in a temporary holding clip, as illustrated in the example of FIG. 2B. Such example implementation can allow placement of the plunger 208, at the time of assembly, at the “empty” position, for example where it is fully bottomed out against the front or proximal end of the barrel/reservoir 204, as illustrated in example of FIG. 2B. A fill port 222 can be connected to the barrel/reservoir 204 either through the main center septum 210 and a cannula 224 (see for example FIG. 3A), or via direct connection to a second port (configured such as, for example, port 202 shown in FIG. 2A), of said barrel/reservoir 204. In a further exemplary implementation, a two-position valve 550 connected to the patient downstream side can be present and, for example, normally closed at time of assembly.

In yet further exemplary implementation, the pump may be filled by pushing the syringe plunger to make it slide on the cable and then the driver can be pulled upon pump initiation to engage the plunger. The engagement with the plunger may be detected by monitoring current draw from motor.

For example, during filling (see FIGS. 2B and 5B) barrel/reservoir 204 can be filled with drug via insert into fill port 222, and a desired amount of fluid can be pushed into fill barrel/reservoir 204. The plunger 208 can be pushed back toward distal end of barrel/reservoir 204, for example only by the filling pressure and by an amount equal to the desired fill volume (see, for example FIG. 2B). When the pump 200 is activated, motor 214/gear 216 can turn the cable spindle/spool 212, 512, 612 and start pulling cable 206. This action can be associated with an observed spike in motor current draw. Once the termination 207/209 of cable 206 reaches the back of the plunger 208 (see for example FIGS. 2C-2E, and compare for example FIGS. 2C and 2D), a second current draw spike can be observed, the latter being larger (for example, much larger) than the initial movement spike. This can indicate an approximate fill volume to a control system, for example 230. In yet further exemplary implementation, a potential encoder on the output gear of the gearbox 216 can be used to correlate cable 206 movement with fill volume.

According to another exemplary non-limiting implementation of a filling feature, the termination of the cable 206 can be fully enclosed in the plunger/stopper 208 (such as insert-molded), and can (for example, due to such configuration) move in unison with the plunger 208 (with some compliance from the materials) (see, for example, FIG. 2A). Such assembled device can, for example, have cable 206 fully spooled, and upon filling, a mechanism can allow cable spool 212, 512, 612 to turn freely until the desired amount of drug is loaded (as illustrated in the examples of FIGS. 2A and 3A). In an exemplary implementation, a potential encoder may provide feedback to the number of spool turns thus giving an estimate of loaded drug. After the device 200 has been filled and the motor 214 is activated, gage spool 212, 512, 612 can be engaged thus allowing cable 206, and thus the plunger/stopper 208, to be pulled and dispense the drug.

According to exemplary implementations, in various configurations a ratchet mechanism may be included to prevent unintended back-driving from increased backpressures. In some cases, depending on the motor/gearbox design, there may be enough back-drive torque that no additional designs would be required to prevent push-back of the piston from backpressure.

According to yet further exemplary implementations, activation of the pump 200 and connection to the patient may be achieved with a secondary port 202/501, for example at a front (proximal end) of the barrel/reservoir 204. This port can be sealed to the outside, for example with a plug/valve such as plug/valve 550, which may comprise an elastomeric seal with an angular notch of suitable width and depth (see, for example, FIGS. 5B and 5C). For example, such notch can serves to allow flow to the patient-side fluid path when the plug/valve 550 is properly rotated, but otherwise be blocking flow. The rotation of the plug/valve 550 may be achieved with a cable or linkage connected to the output spindle/hub via a pin on either component. When the motor 214 starts turning in the pumping direction, the cable/linkage can be pulled and the valve 550 turned opening the fluid path to the patient. The cable or linkage can then be disconnected from the plug/valve 550 and the pumping action can continue.

Exemplary implementation or exemplary embodiments of the disclosure can facilitate at least some of the following non-limiting advantages: simplify pump mechanism and allow medicament deliver systems to use a syringe body; a smaller pump body in the axial direction; a more compact arrangement of mechanism components in the transverse direction potentially providing further space saving; a more drug-friendly syringe concept that avoids cyclical, repetitive motion over the same plastic materials; ease of filling with simple internal components; and potential customization of a spooling action inside the gearbox with potential for further space saving.

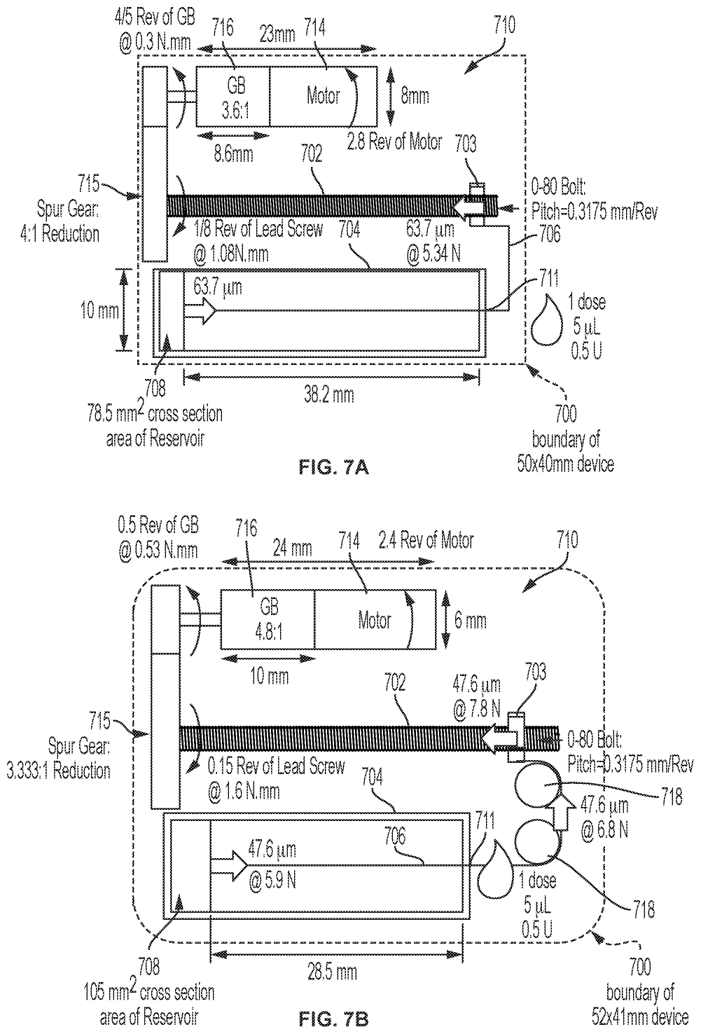

Another exemplary embodiment of the disclosure, as illustrated in block diagrams of FIGS. 7A and 7B (where various numerical dimension are included as illustrative non-limiting examples of relative component sizes and positions), provides a system 710, that can be implemented for example in a devise 700 such as a pump 100 or 200, where a leadscrew 702 can be arranged to pull a nut 703 that in turn pulls a cable 706 that runs through a syringe body (barrel/reservoir) 704 and pulls a plunger 708. The leadscrew 704 can be driven with a gear-train 716 by a motor 714, for example via a spur gear 715. In an exemplary implementation, one or more of each, or both, a pivot pin and/or a pulley 718 can be provided to, for example, guide cable 706, and can be configured, for example between nut 703 and exit point 711 (such as a septum 210) of cable 706 from barrel/reservoir 704. According to exemplary implementations, nut 703 can ride on leadscrew 702 and also be constrained to a purely linear motion, for example by mechanical features such as shafts and bushing/bearings. In a further exemplary implementation, nut 703 can also slide onto, or with respect to, constraining surfaces arranged in a manner that the nut 703 cannot pitch or yaw with respect to axial motion. Addition of a leadscrew configuration can, for example and without limitation, improve control accuracy, defined as degrees of rotation required to deliver a certain dose, by for example increasing the degrees required to turn a lead screw in order to deliver the required volume.

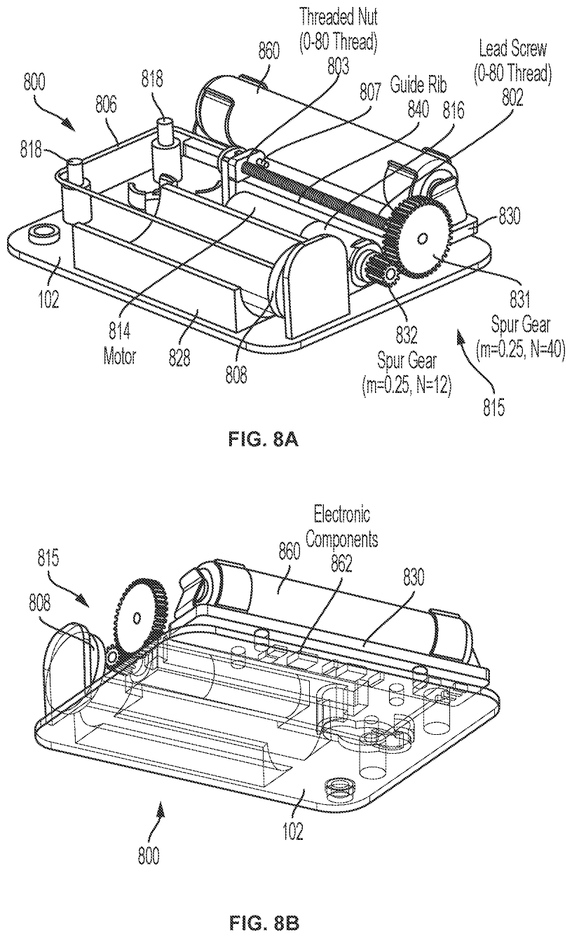

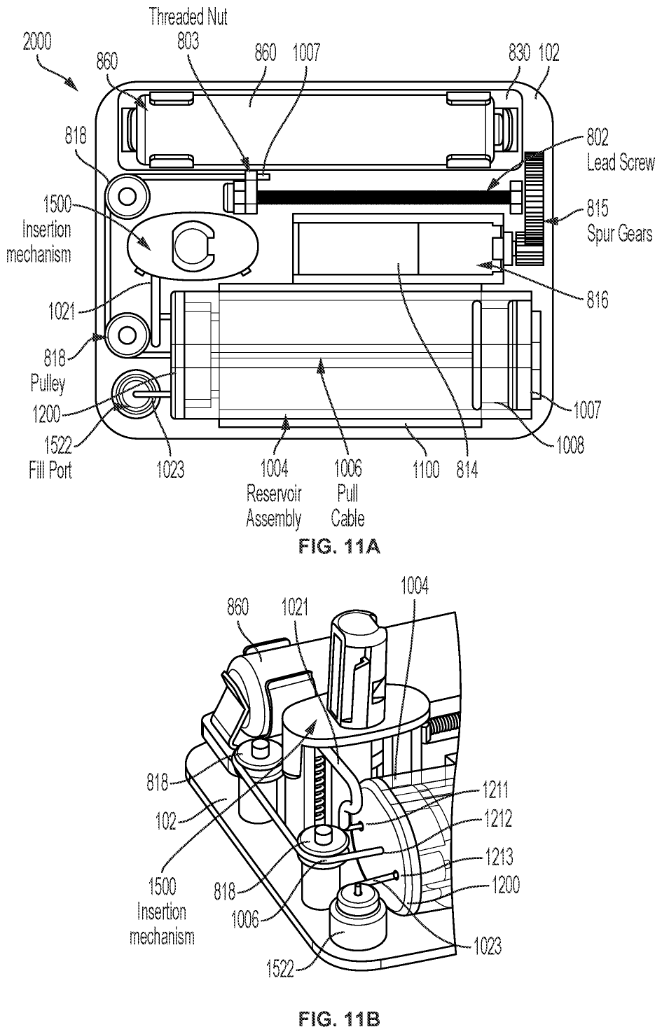

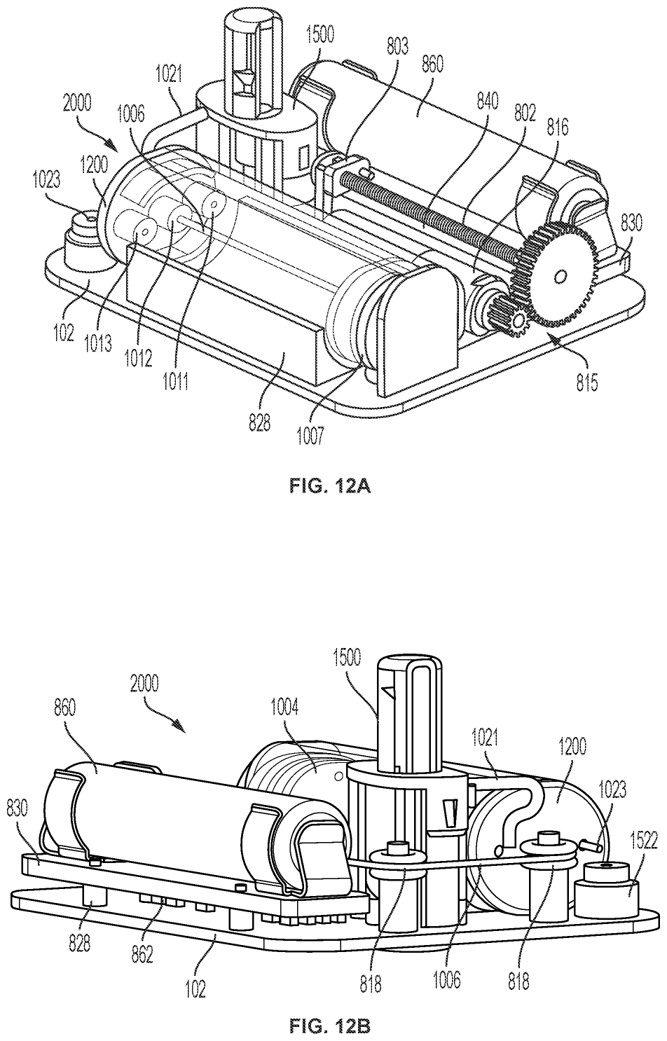

FIG. 8A (where various numerical dimension are included as illustrative non-limiting examples of relative component sizes and positions) illustrates in a perspective view an example of a configuration of at least some of the components a device 800, such as a device or pump 700, according to an exemplary implementation of a medication delivery system implementing a leadscrew design configuration, such as on a base 102 of a wearable disposable patch pump 100, of disclosed exemplary embodiments. As shown in the example of FIGS. 8A and 8B (as well as FIGS. 11A-12B), cable 806 extending from plunger 808 is pulled through a combination of a lead screw 802 and a translation nut 803. A bottom superstructure 828 can be provided on, or integral with, a base of device 800, such as base 102, to support and/or connect, various components disposed thereon.

In an exemplary implementation, a crimp 807 fixes cable 806 to nut 803. A rib guide 840, which can be part of superstructure 828, located on (or integral with) base 102 can be configured to prevent rotation of nut 803 which results in axial translation of nut 803 when lead screw 802 rotates. Spur gears 815 (comprising for example a first gear 831 coaxially connected to lead screw 802 and a second gear 832 connected to gearhead 816) can be used to create a reduction in addition to gearhead 816 of motor 814. Referring to FIG. 8B illustrating a perspective bottom view of device 800, for example a PCB 830 with a battery 860 and electronic components 862 can be configured with respect to base 102, such as, for example, raised off of the bottom of base 102 with battery 860 on top of PCB 830 and electronic components 862 on bottom of PCB 830. Pivot(s) and/or pulley(s) 818, can be provide, for example disposed on base 102, for example to guide cable 806.

Exemplary implementation of exemplary cable-based pump designs disclosed previously herein, which may for example rely on a spool to pull the cable and be efficient and compact, but may require a higher motor control resolution (for example, on the order of single degrees per target dose depending on gearbox used), and may require a relatively higher torque. A possible, but not required, mechanical advantage of a leadscrew design according to exemplary implementations may facilitate reduced torque requirement (for example, by more than an order of magnitude) and similarly the required control angular motion. Such exemplary implementations may use a ball-bearing slide for nut movement control, or may use shafts and bushings.

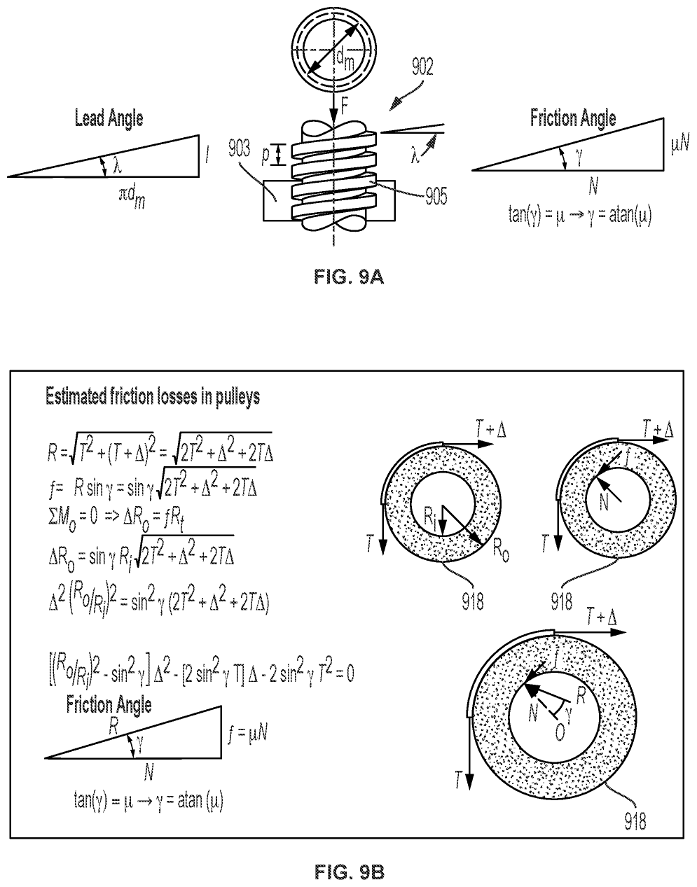

According to an exemplary implementation, a leadscrew design configuration can comprise a leadscrew (for example, having a selected pitch and diameter), bushing/bearings, pulleys, a cable (for example, a low flexural modulus, high tensile Young's modulus wire cable, which can be a braided cable), an optionally applied and/or integrated cable coatings, a gear reduction mechanism, and take into consideration for example cable crimping and/or septum sealing around moving and/or static cable. Referring to example of FIG. 9A, which illustrates a portion of screw 902 (cross-sectional and side views) according to an exemplary implementation, efficiency η of a lead screw 902 having thread 905 can be calculated based on geometry (lead angle λ) and friction (friction angle γ), for example as follows:

$$η = \frac{tan ( λ )}{tan ( λ + γ )}$$

Force F delivered to nut 903 configured on lead screw 902 can be calculated based on efficiency η and input torque T, for example as follows:

$$η = \frac{{W}_{out}}{{W}_{in}} = \frac{Fl}{2 π T} \to F = \frac{2 π T η}{l}$$

FIG. 9B illustrates an example of estimated friction losses computation in pulleys 918, for example by taking into account without limitation factors such as pulley 918 geometry, cable tension, and friction angle.

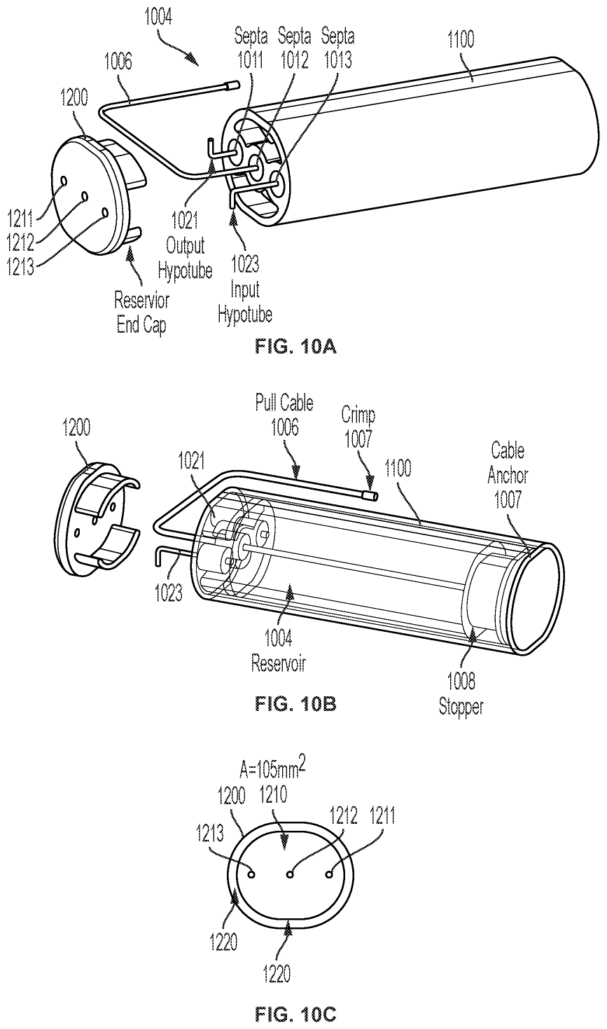

FIGS. 10A and 10B (as well as FIGS. 11B-12B) illustrate in a perspective view an example of a configuration of a barrel/reservoir 1004 of an embodiment that can be implemented in any of the exemplary implementations of disclosed exemplary embodiments of systems, devices, and pumps. While there are a number of different ways that an input tubing, an output tubing, and a cable can interface with a barrel/reservoir, referring to FIG. 10A, according to an exemplary embodiment, barrel/reservoir 1004 comprises body 1100 and three septa 1011, 1012, 1013 assembled into one end, for example a proximal end, of body 1100 of reservoir 1004, which septa can then be compressed by an endcap 1200 (see also FIG. 10C). In an exemplary implementation, septum 1011 can provide access to an output 1021 (for example, via a hypotube) for dispensing drug out of barrel/reservoir 1004), septum 1012 can provide access to cable 1006 connected to plunger/stopper 1008 (and including, for example, cable anchor 1007), and septum 1013 can provide access to an input 1023 (for example, via hypotube) for filling of barre/reservoir 1004 with a drug. In an exemplary implementations, endcap 1200 can have a cross-section area 1210 corresponding to that of cross sectional area of body 1100 of barrel/reservoir 1004, and can include a rim 1220 in contact with proximal rim of body 1100; and can include three openings 1211, 1212, and 1213, for output 1021, cable 1006, and input 1023, respectively. In an exemplary implementation, opening 1211, 1212, and 1213 can be axially aligned with respective access points of septa 1011, 1012, 1013. In an exemplary implementation, endcap 1200 can be ultrasonically/RF wedded to body 1100 of barrel/reservoir 1100.

FIG. 11A illustrates in a top view, and FIGS. 11B, 12A, and 12B in perspective three dimensional views, an example of a configuration of at least some of the components of a device 2000, such as a device or pump 700 and/or 800, according to an exemplary implementation of a medication delivery system implementing a leadscrew design configuration and a barrel/reservoir configuration such as 1004 illustrated in the examples of FIGS. 10A-10C, such as on a base 102 of a wearable disposable patch pump 100, of disclosed exemplary embodiments. As shown in the example of FIGS. 11A-12B, cable 1006 extending from plunger/stopper 1008 (and including, for example, cable anchor 1007) is pulled through septum 1012 via opening 1212 in endcap 1200 of barrel/reservoir 1004 by a combination of a translation, for example threaded, nut 803 and a lead screw 802 driven by motor 814/gearbox 816 (for example, as well as spur gear(s) 815). A bottom superstructure 828 can be provided on, or integral with, a base of device 2000, such as base 102, to support and/or connect, various components disposed thereon, examples of such component are also illustrated in FIGS. 8A, 8B, and 10A-10C.

In an exemplary implementation illustrated in FIGS. 11A-12B, device 2000 comprises an insertion mechanism 1500 for delivering drug from barrel/reservoir 1004 to a patient. Insertion mechanism 1500 can be connected to reservoir 1004 via input 1021 through opening 1211 and septum 1011. On the other hand, in an exemplary implementation, during filling (see also FIGS. 2B and 5B), barrel/reservoir 1004 can be filled with drug by an insert into fill port 1522, such that a desired amount of fluid can be pushed into fill barrel/reservoir 1004 connected to fill port 1522 via input 1023 through opening 1213 and septum 1013.

Further example implementations may use an encoder, for example tied to the plunger/stopper to allow implementation of a full closed loop control, which can improve dose accuracy at nominal and high-backpressure conditions, and which further may allow even higher accuracy feedback to patients.

According to still further exemplary implementations, which can be potentially applicable to any of disclosed exemplary embodiments: an optical or hall-type sensing mechanism, configured for example on a plunger, can be used to detect movement and fill volume. Alternatively or in combination, a plunger can be used as a visual indicator of full volume through a window on the housing 104. Motor load detection when a drive mechanism couples with, and/or actuates movement of, the a plunger can be used to detect fill volume.

While the present disclosure has been shown and described with reference to certain exemplary embodiments thereof, it will be understood by those skilled in the art that various changes in form and details may be made therein without departing from the spirit and scope of the embodiments of the present disclosure. For example, operative variations and alternative different lead designs may be employed to change dosing resolution, encoders may be used to have feedback of drive mechanism, indexing drives can be employed to repeatably and fail-safe advance the plunger. Generally, for example and as noted above, non-circular syringe barrel cross-sections may be employed to optimize space utilization and tailor device size to best suit user comfort. Furthermore, any of the features or elements of any exemplary implementations of the embodiments of the present disclosure as describes above and illustrated in the drawing figures can be implemented individually or in any combination(s) as would be readily appreciated by skilled artisans without departing from the spirit and scope of the embodiments of the present disclosure.

In addition, the included drawing figures further describe non-limiting examples of implementations of certain exemplary embodiments of the present disclosure and aid in the description of technology associated therewith. Any specific or relative dimensions or measurements provided in the drawings other as noted above are exemplary and not intended to limit the scope or content of the inventive design or methodology as understood by artisans skilled in the relevant field of disclosure.

Other objects, advantages and salient features of the disclosure will become apparent to those skilled in the art from the details provided, which, taken in conjunction with the annexed drawing figures, disclose exemplary embodiments of the disclosure.

1. A system comprising:
an output for connection to a medication delivery mechanism;
a container for said medication in fluid communication with said output;
a plunger disposed in said container and having a first surface in communication with said medium, said first surface facing a proximal end of said container; and
a cable connected to said plunger and extending from said first surface of said plunger toward said proximal end of said container and exiting out of said container via said proximal end of said container.

2. The system of claim 1 wherein, when said plunger is positioned at a distance from said proximal end of said container, pulling of said cable out of said container causes axial displacement of said plunger with respect to said container toward said proximal end of said container, said axial displacement of said plunger causing delivery of said medication.

3. The system of claim 2, further comprising:
a spool; and
a motor for rotating said spool in a first rotational direction,
wherein said cable is connected to said spool at a connection point, and rotation of said spool winds said cable onto said spool causing said pulling of said cable.

4. The system of claim 2, further comprising:
a lead screw;
a nut in threaded communication with said lead screw; and
a motor for rotating said lead screw,
wherein said cable is connected to said nut at a connection point, and rotation of said lead screw axially displaces said nut causing said pulling of said cable.

5. The system of claim 3, further comprising a gear mechanism transferring rotation of said motor to said spool.

6. The system of claim 4, further comprising a gear mechanism transferring rotation of said motor to said lead screw.

7. The system of claim 3, further comprising at least one pivot pin and/or pulley configured between said proximal end of said container and said connection point.
wherein said at least one pin and/or pulley contacts said cable to guide said cable between said proximal end of said container and said connection point.

8. The system of claim 7, wherein a configuration of said at least one pivot pin and/or pulley adjusts tension of said cable.

9. The system of claim 1, further comprising a septum configured at said proximal end of said container,
wherein said septum comprises said output for connection to said medication delivery mechanism, said septum providing leak proof communication between said output and said container.

10. The system of claim 1, further comprising a septum configured at said proximal end of said container,
wherein said cable exits said container via said septum, and said septum provides leak proof communication between said cable and said container.

11. The system of claim 1, further comprising a fill port in fluid communication with said container at said proximal end of said container.

12. The system of claim 11, further comprising a septum configured at said proximal end of said container, wherein said cable exits said container via said septum, and said septum provides leak proof communication between said fill port and said container.

13. The system of claim 11, wherein
when said plunger is positioned at said proximal end of said container, filling of said container via said fill port causes axial movement of said plunger toward a distal end of said container.

14. The system of claim 13, wherein said cable is removably anchored at said distal end of said container, and said plunger axially slides on said cable with respect to said container during said filling.

15. The system of claim 3 wherein said spool comprises a clutch mechanism for engaging said spool to rotate in said first rotational direction with said motor, and disengaging said spool from said motor to allow rotation of said spool in a second rotational direction opposite said first rotational direction.

16. The system of claim 15 further comprising a fill port in fluid communication with said container at said proximal end of said container
when said plunger is positioned at said proximal end of said container, said clutch mechanism disengages said spool from said motor, and
filling of said container via said fill port causes axial movement of said plunger toward a distal end of said container causing unwinding of said cable off said spool.

17. The system of claim 5 wherein said spool comprises a clutch mechanism for engaging said spool to rotate in said first rotational direction with said motor, and disengaging said spool from said motor to allow rotation of said spool in a second rotational direction opposite said first rotational direction.
